(19) United States
(12) Patent Application Publication
(10) Pub. No.: US 2003/0036077 A1
(43) Pub. Date: Feb. 20, 2003

(54) Methods for generating an mRNA expression profile from an ecellular mRNA containing blood sample and using the same to identify functional state markers

(75) Inventors: Alex Chenchik, Palo Alto, CA (US); Leonid Iakoubov, Pleasanton, CA (US); Howard B. Urnovitz, San Francisco, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Chronix Biomedical, Benecia, CA (US)

(21) Appl. No.: 10/161,101

(22) Filed: May 31, 2002

Related U.S. Application Data
(60) Provisional application No. 60/327,565, filed on May 31, 2001.

(51) Int. Cl. C12Q 1/68; C07H 21/02; C12P 19/34

(52) U.S. Cl. 435/6; 536/23.1; 536/24.3; 435/91.2

(57) ABSTRACT

Methods for generating an mRNA expression profile are provided. In the subject methods, a population of nucleic acid targets is first generated from an acellular blood sample that contains a plurality of distinct mRNAs, i.e., a disease specific particular blood fraction. The resultant nucleic acid targets are hybridized to an array of nucleic acid probes to obtain an mRNA expression profile. The subject mRNA expression profiles are useful in the identification of disease specific markers. In such applications, the mRNA expression profiles are compared to a control expression profile to identify disease specific markers, where the identified markers subsequently find use in diagnostic applications. The subject methods also find use in diagnostic applications, where the mRNA expression profile is compared to a reference in making a diagnosis of the presence of a disease condition. Finally, kits for use in practicing the various methods are provided.

### TECHNICAL FIELD

The field of this invention is diagnostics, particularly blood dependent diagnostics, including prognostic and predictive diagnostics.

### BACKGROUND OF THE INVENTION

Diagnostic procedures are evaluations that identify the presence of a certain condition or functional state of an organism, e.g., a disease state or condition, in a subject based on one or more observed parameters, e.g., symptoms, markers or analytes, etc. Many diagnostic procedures currently rely on the identification of certain disease- and disease condition-related analytes or markers. In many diagnostic procedures, a body derived sample, e.g., blood or fraction thereof, tissue or sample prepared therefrom, etc., is assayed for the presence of the marker or analyte.

A desirable sample to analyze in diagnostic procedures is blood or a fraction/preparation thereof because such samples can be obtained in a relatively minimally invasive manner, as compared with procedures requiring the use of a tissue biopsy derived sample. Furthermore, blood based diagnostic procedures can often detect the presence of a disease condition early in the progression of a disease, often leading to more effective treatment protocols.

Despite the advantages promised by blood based diagnostic procedures, as of today, the diagnostics of many diseases cannot be done by blood analysis and require the use of more invasive procedures to obtain the requisite sample. In addition, most of the to date developed blood diagnostic assays do not target such important questions as disease stage, prognosis, individual predictive therapy, etc. To overcome the above problems, new blood markers reliably correlated with various diseases, disease status or other physiological states, for example, disease susceptibility, stress, etc., must be identified.

As such, there continues to be great demand for technology which will allow one to perform high throughput discovery of novel blood markers for multiple diseases and functional states.

Relevant Literature

Of interest are U.S. Pat. No. 5,972,615 and PCT publications WO 99/49083; WO 98/24935; and WO 97/35589. See also, WO 97/35589; Wieczorek, et al., “Isolation and characterization of an RNA-proteolipid complex associated with the malignant state in humans,” Proc. Natl. Acad. Sci. (1985) 82:3455-3459; Ceccarini, et al., “Biochemical and NMR studies on structure and release conditions of RNA-containing vesicles shed by human colon adenocarcinoma cells,” Int. J. Cancer (1989) 44:714-721; Umovitz et al., “RNAs in the sear of Persian Gulf War veterans have segments homologous to chromosome 22a11.2,” Clin. Diagn. Lab. Immunol. (1999) 6:330-335; Kopreski, et al., “Detection of tumor messenger RNA in the serum of patients with malignant melanoma,” Clin. Cancer Res. (1999) 5:1961-1965; Kopreski, et al., “Cellular-versus extracellular-based assays. Comparing utility in DNA and RNA molecular marker assessment,” Ann. N.Y. Acad. Sci. (2000) 906:124-128; and Hasselmann, et al., “Detection of tumor-associated circulating mRNA in serum, plasma and blood cells from patients with disseminated malignant melanoma,” Oncol. Rep. (2001) 8:115-118.

### SUMMARY OF THE INVENTION

Methods for generating an mRNA expression profile are provided. In the subject methods, a population of nucleic acid targets is first generated from an acellular blood sample, particularly a specific particular blood fraction (SPBF), that contains a plurality of distinct mRNAs, typically functional state, e.g., disease condition, markers. Use of the SPBF, as opposed to a total blood acellular mRNA sample, is an important feature of the subject invention. The nucleic acid targets generated from the SPBF are then hybridized to an array of nucleic acid probes to obtain an mRNA expression profile. The subject mRNA expression profiles produced using the subject methods are useful in a number of different applications, including the identification of disease specific markers. In such applications, the mRNA expression profiles are compared, e.g., visually, by querying a database, etc., to a control expression profile, e.g., an expression profile obtained from a normal individual or a composite expression profile, to identify functional state, e.g., disease, specific markers, where the identified markers subsequently find use in diagnostic applications, including but not limited to: predicting of disease susceptiblity, disease identification, prognosis, predicting optimal therapy, disease progress monitoring, disease therapy monitoring, etc. Other applications in which the subject profiles find use include the above diagnostic applications, where the mRNA expression profile is compared to a reference in making a diagnosis of the presence of a disease condition, and disease management applications, in which the progression of a disease state is monitored by monitoring changes in an mRNA expression profile. Finally, kits for use in practicing the various methods are provided.

### DEFINITIONS

The terms “plasma” and “serum,” mean relatively cell-free blood obtained as a result of low speed (up to 800×g) centrifugation. These acellular blood fractions have a very complex composition. Plasma and serum have a soluble fraction which is comprised by soluble proteins, lipids, nucleic acids (DNA and RNA), polysaccharides, proteoglycans, and other low and high-molecular weight molecules and complexes between these molecules, like RNA-lipid, RNA-protein, nucleoproteids, RNA-proteolipid complexes, etc. There are also multiple higher molecular weight plasma constituents that can be considered for simplicity as the insoluble fraction and can be separated from the soluble fraction by high-speed centrifugation (usually at 100,000×g for 2 hr). This “insoluble fraction” is also fairly heterogeneous and is made up of contaminating cells from the cellular fraction, different size apoptotic bodies, cell debris (portions of destroyed or damaged cells), vesicles, microvesicles, particles, ectosomes, exosomes, secretory vesicles, nucleosome-like structures, virus-like structures, etc.

The “SPBF” or “specific particular blood fraction” from which the target nucleic acids are prepared in the subject methods of generating mRNA expression profiles is a specific particle containing fraction of blood that is an acellular blood sample which includes a plurality of distinct mRNAs that differ from each other by sequence. The subject SPBF employed in the subject methods is a specific particle containing fraction of plasma that may be isolated in a preferred embodiment by centrifugation between 2,000×g and 20,000×g, and preferably between 4,000×g and 10,000×g (see Table 1, infra). A representative centrifugation protocol suitable for use in preparation of the SPBF is reported in the experimental section, infra, where any centrifugation or other blood fractionation protocol capable of producing an SPBF that is substantially the same as the fraction produced using the centrifugation protocol reported herein may be employed. The terms “probe” and “target” are used herein and in accordance with the Nature Genetics Supplement, Vol. 21, published January 1999, such that the term “probe” refers to the “tethered” nucleic acid of an array, i.e., the nucleic acid immobilized to the surface of the array substrate, while the term “target” refers to the nucleic acid in solution with which the array is contacted during use.

The “functional state” means the condition of the host, e.g. whether the host is under stress, afflicted with a particular disease condition, the age of the host, etc, and therefore includes within its scope both disease related and disease specific conditions, as well as other conditions. The term “disease-related” is broader than “disease-specific.” In addition to the elements specific for the disease pathogenesis which are “disease-specific,” the former term also refers to additional elements related to a disease condition, e.g., how the host immune system is reacting to the disease state, the state of host, e.g., in terms of stress, circadian rhythms, toxicity exposure, etc. The relevant host can be human or non-human, e.g., an animal model, such as a mouse, rat, etc., for human functional state, e.g., disease, of interest.

### BRIEF DESCRIPTION OF THE FIGURES

FIG. 1 provides expression profiles generated from a variety of different blood fractions, including the subject disease specific particular blood fractions.

FIG. 2 provides expression profiles generated from the disease specific particular blood fraction of a subject suffering from meyloma and a healthy control subject.

FIG. 3 provides Tables 1a and 1b referenced in Example 5.

### DESCRIPTION OF THE SPECIFIC EMBODIMENTS

Methods for generating an mRNA expression profile are provided. In the subject methods, a population of nucleic acid targets is first generated from an SPBF. The subject mRNA expression profiles produced using the subject methods are useful in a number of different applications, including the identification of disease specific markers. In such applications, the mRNA expression profiles are compared, e.g., visually, by querying a database, etc., to a control expression profile, e.g., an expression profile obtained from a normal individual or a composite expression profile, to identify functional state, e.g., disease, specific markers, where the identified markers subsequently find use in diagnostic applications, including but not limited to: predicting of disease susceptiblity, disease identification, prognosis, predicting optimal therapy, disease progress monitoring, disease therapy monitoring, etc. Other applications in which the subject profiles find use include the above diagnostic applications, where the mRNA expression profile is compared to a reference in making a diagnosis of the presence of a disease condition, and disease management applications, in which the progression of a disease state is monitored by monitoring changes in an mRNA expression profile. Finally, kits for use in practicing the various methods are provided.

In further describing the subject invention, the methods of obtaining mRNA expression profiles are first described in greater detail. Next, the use of the subject mRNA expression profiles in the identification of functional state, e.g., disease specific and/or disease related markers is described, as well as the other representative applications mentioned above, e.g., diagnostic and disease progression monitoring applications. Finally, the use of the identified functional state markers in diagnostic applications is reviewed.

Before the subject invention is further described, it is to be understood that the invention is not limited to the particular embodiments of the invention described below, as variations of the particular embodiments may be made and still fall within the scope of the appended claims. It is also to be understood that the terminology employed is for the purpose of describing particular embodiments, and is not intended to be limiting. Instead, the scope of the present invention will be established by the appended claims.

In this specification and the appended claims, the singular forms “a,” “an” and “the” include plural reference unless the context clearly dictates otherwise. Unless defined otherwise, all technical and scientific terms used herein have the same meaning as commonly understood to one of ordinary skill in the art to which this invention belongs.

Methods of Generating mRNA Expression Profiles

As summarized above, the subject invention is directed to particular methods of generating mRNA expression profiles. As is known in the art, mRNA expression profiles are generated by preparing a collection of target nucleic acids from an initial sample, e.g., via template driven nucleic acid synthesis protocols, followed by contact of the generated target population with an array of probe nucleic acids under hybridization conditions, which step results in the generation of an mRNA expression profile made up of a plurality of probe-target duplex structures on the surface of the array. A feature of the subject invention is that a particular blood fraction is employed as the sample from which the target nucleic acids are prepared.

Production of Target Nucleic Acids

As indicated above, the first step in the subject methods is to produce a population of target nucleic acids. The first part of this step is to obtain the SPBF sample. Next, the target nucleic acids are generated from the obtained SPBF, e.g., using a template driven nucleic acid synthesis protocol. Each of these steps is now further described in greater detail.

SPBF Procurement

As defined above, the “SPBF” or “specific particular blood fraction” from which the target nucleic acids are prepared in the subject methods of generating mRNA expression profiles is a specific particle containing fraction of blood that is an acellular blood sample which includes a plurality of distinct mRNAs that differ from each other by sequence. The subject SPBF employed in the subject methods is a specific particle containing fraction of plasma that may be isolated in a preferred embodiment by centrifugation between 2,000×g and 20,000×g, and preferably between 4,000×g and 10,000×g (see Table 1, infra). A representative centrifugation protocol suitable for use in preparation of the SPBF is reported in the experimental section, infra, where any centrifugation or other blood fractionation protocol capable of producing an SPBF that is substantially the same mRNA composition as the fraction produced using the centrifugation protocol reported herein may be employed. As such, while the SPBF is defined herein in terms of the manner in which it is produced via centrifugation protocols, the SPBF may be produced using any convenient technique, so long as the constituents of interest are present in the blood fraction produced by the employed protocol, e.g., a plurality of mRNA species present in a quantity sufficient to generate nucleic acid target for use in gene expression analysis.

The use of the above described SPBF of blood is an important feature of the subject invention. The use of this specific blood fraction is important because other blood fractions, e.g., total plasma and/or serum, and other plasma or serum fractions obtainable by differential centrifugation, are not preferred for use in the subject methods for the following reasons. Contrary to the indicated preferred 4,000-10,000×g fraction, the fraction obtained between 300-800×g (usually used for separating plasma fraction from blood cells) and 4,000×g is not disease- or functional state-specific since the disease- or other functional state specific component is masked by mRNA molecules originating from platelets, other cell originated contaminants and debris of normally dying, apoptotic and/or destroyed blood cells. Alternatively, the fraction of plasma that can be isolated between 20,000×g and 100,000×g contains mainly DNA and significantly reduced amounts of mRNA and is therefore not useful for expression profiling applications. The particle-free fraction (obtained by higher than 100,000×g) contains only trace amounts of RNA, since soluble RNA is not protected from enzyme (nuclease, RNase) degradation.

The preferred SBPF employed in the subject methods, e.g., the 4,000×g-10,000×g fraction obtained from differential centrifugation, as described in the experimental section, infra, contains undegraded mRNA and substantially low amounts of DNA. By substantially low amounts of DNA is meant that the amount of DNA in this particular fraction does not exceed about 10%, usually does not exceed about 5% and more usually does not exceed about 1% (by wt.) of RNA amount purified from disease specific fraction. The yield of RNA from this fraction is only 1-10 ng per ml of plasma which is about 0.1% of whole RNA that is contained in plasma isolated by using standard protocol. With respect to the mRNA component, the mRNA component is made up of a plurality of a number of different mRNA molecules that differ from each other in terms of sequence, where the number of mRNA molecules is at least about 500, usually at least about 1,000 and more usually at least about 2,000 and may be much higher. RNA purified from SPBF has a rather high complexity similar to cellular total RNA and comprises substantially non-degraded polyadenylated mRNA, non polyadenylated mRNA molecules and other RNA molecules, like tRNA, rRNA, etc. The mRNA molecules encode proteins endogenous to the subject or host from which the sample is obtained, and as such are transcribed from host genomic material. Since the subject is a human in many embodiments, the mRNA molecules of interest encode human proteins and are transcribed from human genomic nucleic acids.

In addition to the above described mRNA component, the preferred 4,000×g-10,000×g disease specific blood fraction, i.e., SPBF, also typically includes particles that are smaller than cells, i.e., particles that do not exceed about several microns in diameter (e.g., 3-5 μM) but have a diameter that is greater than 0.05-0.1 μM, where the particles typically range between about 0.15 and 2.0 μM and more typically range in diameter between about 0.2 and 1 μM. These sub-cellular particles have a complex composition that is made up of undegraded mRNA, as described above, as well as proteins, lipids, sugars and other molecules, where the particles may or may not be substantially free of DNA, where when present DNA is present as a contaminant. The subject particles may include proteins expressed by mRNA molecules present in the particles, i.e., the mRNA component of the particles may at least partially correspond to protein composition of the particles. In other words, at least some of the proteins in the particle fraction of interest may be encoded by mRNA molecules also present in the particle fraction of interest.

As mentioned above, the SPBF that is employed in the subject methods of generating mRNA expression profiles may be obtained using any convenient methodology. In one representative protocol, differential centrifugation is employed to obtain the disease specific particular blood fraction, which is a fraction that is present between 2,000 and 40,000×g, more preferably between about 4,000 and 20,000×g and more usually between about 4,000 and 10,000×g. In this representative protocol, an initial blood sample from a subject, e.g., patient, is first obtained or drawn, typically by a standard methods such as via collection tubes or vacutainers with anticoagulant for preparation of serum or as in a preferred embodiment with anticoagulant, like EDTA, sodium citrate, and the like for preparation of plasma. The resultant obtained blood sample is then fractionated to obtain a fresh plasma fraction, e.g., via centrifugation (for example at 800×g for 10 min) followed by plasma fraction collection. Such methods are known and readily practiced by those of skill in the art.

Following obtainment of the initial plasma fraction, the SPBF, as described above, is obtained. The initial plasma fraction may be used immediately upon its production or after the plasma fraction has been stored for a certain period of time prior to use. Where the plasma fraction has initially been stored in liquid form, it is preferably refrigerated and stored at 0-4° C. for up to 24 hours. Where the plasma fraction is stored in frozen form, the frozen plasma fraction is preferably stored at −20 to −70° C., preferably at −70° C., for up to about 2-3 years.

The plasma fraction, following a thawing step where necessary, is centrifuged at 4,000×g for 30 min at 4° C. and the resultant supernatant again centrifuged at 20,000×g for 30 min at 4° C. (The above specific parameters are merely representative and should not be construed as limiting the protocol employed to produce the SPBF). The resultant precipitate that is collected after this centrifugation is the SPBF of interest that is employed in the subject methods. It should be understood that the conditions for centrifugation (speed, time and temperature) could vary and depends on volume of plasma used, type of centrifuge, etc., and should be optimized in some cases. As such, the above specific parameters are merely representative. Usually for the clinical setting the volume of plasma can be between 100 μl and 50 ml, more commonly between 200 μl and 10 ml and for most applications between 0.5 ml and 5 ml. The disclosed protocol works efficiently for 0.5-1 ml of starting blood volume but can be optimized for smaller amounts of plasma samples.

Target Generation

Following SPBF procurement, as described above, the second step is to produce a population of target nucleic acids from this initial SPBF. In this step of the subject methods, total RNA or its transcriptionally active fraction mRNA can be isolated from the disease specific particular blood fraction and labeled and used directly as a target nucleic acid, or it may be converted to a labeled cDNA, cRNA, etc. via methods such as reverse transcription, transcription and/or PCR. In many embodiments the test target nucleic acids are generally isolated from the SPBF and then converted to other nucleic acids using technology known to and readily practiced by those of skill in the art, such as PCR, reverse transcription, transcription, generating complementary nucleic acid target by hybridization, etc., e.g. mRNA, cDNA, PCR products, cRNA, oligonucleotides, and the like.

In certain embodiment, the methods of target nucleic generation will employ the use of oligonucleotide primers in template (for example mRNA) dependent primer extension reactions, where the primers can be anchored by bacteriophage RNA polymerase promoter. The primers may be designed to copy a large spectrum of RNA species, e.g. oligo(dT) primers or random primers, e.g., hexamers, or designed to specifically copy a subset of genes of interest, i.e., gene specific primers. In a preferred embodiment of the subject invention, the test target nucleic acid sequences are generated using a set of a representative number of gene specific primers, as described in U.S. Pat. No. 5,994,076; the disclosure of which is incorporated herein by reference. After the copying step, i.e., conversion of mRNA to cDNA, cDNA can be amplified by PCR or by linear amplification using bacteriophage RNA polymerase mediated transcription.

In an alternative embodiment, the initial mRNA population is contacted with a control set of target nucleic acids as described in U.S. application Ser. No. 09/750,452, the disclosure of which is herein incorporated by reference, where the control set of target nucleic acids is made up of a plurality of distinct nucleic acids of known sequence, where each distinct nucleic acid is present in a known amount. The particular nucleic acids present in the control set are those that correspond to the genes to be assayed, e.g., those that hybridize under stringent conditions to mRNAs of the same genes that are to be probed in a given assay. For example, in a protocol where the expression of 500 different genes is to be assayed using an array displaying 500 different probes (one corresponding for to each probe on the array), one for each gene to be assayed, the control set that is contacted with the mRNA sample includes 500 different control target nucleic acids for which the sequence and amount of each constituent nucleic acid member is known, e.g., where all of the different control target nucleic acids are present in equimolar amounts in the control set.

Contact under stringent hybridization conditions results in the production of a population of single stranded nucleic acids and duplex structures of mRNAs hybridized to their complementary control target nucleic acids present in the initial control set of target nucleic acids. These duplex structures are then separated from the single stranded nucleic acids present in the hybridization mixture, which components include non-hybridized mRNAs present in the original sample, non-hybridized control target nucleic acids present in the original control set, etc. Separation may be by any convenient means, including separation based on physical criteria, e.g., size separation such as by electrophoresis, chromatography, e.g., using oligo dT beads which bind complex polyA+ RNA with hybridized control targets (as exemplified in the Experimental Section, infra), centrifugation, selective precipitation, etc. Alternatively, chemical separation means, e.g., chemical crosslinking or modification of single stranded or double stranded fraction, enzymatic separation means, etc., may be employed. For example, an enzyme or enzyme mix that degrades single stranded nucleic acids but not double stranded nucleic acids, e.g., one or more single stranded nucleases, may be employed, where representative enzymes of interest include, but are not limited to: ribonuclease A, −T1, −B, −I, mung bean nuclease, S1 nuclease; and the like.

In many embodiments, the target nucleic acids generated in this step of the subject methods are labeled target nucleic acids. Labeled target nucleic acids can be provided in any convenient manner. In certain embodiments, PCR is carried out in the presence of labeled dNTPs such that the resultant, amplified cDNA is labeled and serves as the labeled or target nucleic acid. Labeled nucleic acids can also be produced by carrying out PCR in the presence of labeled primers, where either or both of the CAPswitch oligonucleotide complementary primer and anchor sequence complementary primer may be labeled. In yet an alternative embodiment, instead of producing labeled amplified cDNA, one may generate labeled RNA from the amplified ds cDNA, e.g. by using an RNA polymerase such as E.coli RNA polymerase, or other RNA polymerases requiring promoter sequences, where such sequences may be incorporated into the arbitrary anchor sequence. Labeled nucleic acid can also be produced by contacting the resultant amplified cDNA with a set of gene specific primers, a polymerase and dNTPs, where at least one of the gene specific primers and/or dNTPs are labeled. In this embodiment, one of either the gene specific primers or dNTPs, preferably the dNTPs, will be labeled such that the synthesized nucleic acid targets are labeled.

By labeled is meant that the entities comprise a member of a signal producing system and are thus detectable, either directly or through combined action with one or more additional members of a signal producing system. Examples of directly detectable labels include isotopic and fluorescent moieties incorporated into, usually covalently bonded to, a nucleotide monomeric unit, e.g. dNTP or monomeric unit of the primer. Isotopic moieties or labels of interest include ³²P, ³³P, ³⁵S, ¹²⁵I, ³H, and the like. Fluorescent moieties or labels of interest include coumarin and its derivatives, e.g. 7-amino-4-methylcoumarin, aminocoumarin, bodipy dyes, such as Bodipy FL, cascade blue, fluorescein and its derivatives, e.g. fluorescein isothiocyanate, Oregon green, rhodamine dyes, e.g. texas red, tetramethylrhodamine, eosins and erythrosins, cyanine dyes, e.g. Cy3 and Cy5, macrocyclic chelates of lanthanide ions, e.g. quantum dye™, fluorescent energy transfer dyes, such as thiazole orange-ethidium heterodimer, TOTAB, etc. Labels may also be members of a signal producing system that act in concert with one or more additional members of the same system to provide a detectable signal. Illustrative of such labels are members of a specific binding pair, such as ligands, e.g. biotin, fluorescein, digoxigenin, antigen, polyvalent cations, chelator groups and the like, where the members specifically bind to additional members of the signal producing system, where the additional members provide a detectable signal either directly or indirectly, e.g. antibody conjugated to a fluorescent moiety or an enzymatic moiety capable of converting a substrate to a chromogenic product, e.g. alkaline phosphatase conjugate antibody; and the like. Another example of a labeling protocol of interest is that disclosed in U.S. patent application Ser. No. 09/411,351, the disclosure of which is herein incorporated by reference.

Using the above protocols, a population of target nucleic acids, which may or may not be labeled depending on the detection protocol employed in the subject methods, is produced. As mentioned above, this population of target nucleic acids is a mirror of the mRNA profile of the starting disease specific particular blood fraction that is used to generation the target nucleic acids. Since it is a mirror of this initial mRNA profile, the sequence of each of the different nucleic acids in the population of target nucleic acids corresponds to a sequence of an mRNA molecule in the initial disease specific particular blood fraction. By corresponds is meant that the sequence is the same as the complement of a sequence of an RNA molecule found in the initial sample, or the sequence is the same as or the complement of the sequence of a first strand cDNA that is reverse transcribed from an RNA molecule found in the initial sample. In addition, since the population of target nucleic acid mirrors the initial mRNA profile, the abundance of each target nucleic acid is proportional to the abundance of each of the corresponding mRNAs in the initial sample, such that the abundance of each of the initial mRNAs in the sample is reflected in the final target nucleic acid population.

Expression Profile Generation

As mentioned above, the population of target nucleic acids produced above is a representation of the mRNA profile of the SPBF from which the labeled nucleic acids are generated. The next step in the subject methods is to derive from this resultant complex mixture of target nucleic acids the sequence and amount of each constituent member of the mixture, or at least a representative proportion thereof (e.g., 50%, 40%, 30%, 20%, 10%, 5%) to derive an mRNA expression profile, which expression profile, in the broadest sense, can be viewed a set of data points that provides the amount and sequence of each different type of nucleic acid in the population of target nucleic acids. Amount can refer to an absolute quantity or relative quantity, as explained in greater detail infra.

This step of generating the mRNA expression profile typically comprises separating the different types of target nucleic acids from each other based on sequence and then quantitating each different type of target nucleic acid. Separation of the different target nucleic acids can be accomplished in a number of different ways. Where one knows that the target nucleic acids of the set differ by size, size fractionation protocols may be employed, e.g., electrophoretic separation protocols may be employed. The resultant pattern of resolved bands in the gel following an electrophoretic separation represents an mRNA expression profile. See Liang & Pardee, Science (1992) 257: 967. In another approach, the target nucleic acids (either fragments or full-length) can be cloned and sequences from cDNA libraries, e.g., by SAGE (serial analysis of gene expression). Alternatively and in many preferred embodiments, the mRNA expression profile is produced using an array of probes immobilized on the surface of a solid support, as described in greater detail below.

As mentioned above, separation using arrays of probe nucleic acids immobilized to the surface of a solid support is a preferred means of separating the target nucleic acids according to the subject invention. In these embodiments, the complex mixture of target nucleic acids is typically contacted with the array of immobilized probe nucleic acids under hybridization conditions (typically stringent hybridization conditions) and the presence of duplex structures on the array surface is subsequently detected to obtain the desired expression profile.

A variety of nucleic acid arrays are known in the art and may be used in the subject methods. The nucleic acid arrays employed in the subject methods typically have a plurality of nucleic acid probe spots, and preferably in many embodiments oligonucleotide or polynucleotide probe spots, stably associated with or immobilized on a surface of a solid support, where the solid support may be rigid, e.g. glass, or flexible, e.g. nylon membrane or plastic film. At least a portion of the nucleic acid spots on the array are made up of probe nucleic acids. Arrays that may be used in the subject methods include, but are not limited to: nucleic acid biochips, e.g. cDNA biochips, RNA biochips, polynucleotide biochips, oligonucleotide biochips, and the like. Of particular interest are the arrays described in: U.S. Pat. Nos. 5,994,076 and 6,087,102; and U.S. patent application Ser. Nos. 09/053,375; 09/104,179; 09/440,829 and 09/752,293; the disclosures of which are herein incorporated by reference.

The target nucleic acids are hybridized to the array by contacting them to the array under hybridization conditions. By “hybridization conditions” is meant conditions sufficient to promote Watson-Crick hydrogen bonding between the target and probe nucleic acids. The hybridization conditions, such as hybridization time, temperature, wash buffers used, etc. can be altered to optimize the efficient and specific binding of the target sequences. Test target nucleic acids having sequence similarity to the probes may be detected by hybridization under low stringency conditions, for example, at 50° C. and 6×SSC (0.9 M sodium chloride/0.09 M sodium citrate, 1% SDS) and remain bound when subjected to washing at 55° C. in 1×SSC (0.15 M sodium chloride/0.015 M sodium citrate, 1% SDS). Test target sequences with sequence identity may be determined by hybridization under stringent conditions, for example, at 60° C. or higher and 6×SSC (0.9 M sodium chloride/0.09 M sodium citrate, 1% SDS). Another example of stringent hybridization conditions is overnight incubation at 42° C. in a solution: 50% formamide, 5×SSC (150 mM NaCl, 15 mM trisodium citrate), 50 mM sodium phosphate (pH7.6), 5×Denhardt's solution, 10% dextran sulfate, and 20 μg/ml denatured, sheared salmon sperm DNA, followed by washing the filters in 0.1×SSC at about 65° C. Stringent hybridization conditions are hybridization conditions that are at least as stringent as the above representative conditions. Other stringent hybridization conditions are known in the art, see e.g., Maniatis et al., and in PCT WO 95/21944. Preferably, the control target nucleic acids have a region of substantial identity to the provided probe sequences on the array, and bind selectively to their respective probe sequences under stringent hybridization conditions.

Following hybridization, e.g., under stringent hybridization conditions, non-hybridized labeled nucleic acid is removed from the support surface, conveniently by washing, generating a pattern of hybridized nucleic acids or duplex structures on the substrate surface. A variety of wash solutions and protocols are known to those of skill in the art and may be used. See Sambrook, Fritsch & Maniatis, Molecular Cloning: A Laboratory Manual (Cold Spring Harbor Press)(1989). Where the target nucleic acids are unlabeled prior to contact with the array, a post contact labeling step may be employed to provide for visualization and detection of duplex structures on the array surface. In these embodiments, a sandwich format may be employed in which the target nucleic acids are hybridized to a second labeled nucleic acid complementary to a single stranded portion of the hybridized target nucleic acid, e.g., the gene specific portion of the target nucleic acid, which produces detectably labeled sandwich structures on the array surface. See e.g., Maldonado-Rodriquez et al., Mol. Biotechnol. (1999) 11:1-12.

The resultant hybridization patterns of duplex structures may be visualized or detected in a variety of ways, with the particular manner of detection being chosen based on the particular label being employed, e.g., label of the target nucleic acid, where representative detection means include scintillation counting, autoradiography, fluorescence measurement, calorimetric measurement, light emission measurement, light scattering and the like.

Following separation, e.g., via hybridization to an array of probe nucleic acids as described above, the amount of each type of target nucleic acid is determined, where the amount may be determined in relative or absolute terms, as is known in the art. See e.g., U.S. Pat. No. 6,040,138, the disclosure of which is herein incorporated by reference. Levels of hybridization of test target RNA to the probe compositions can be standardized by comparing the hybridization signal of the test with control target sequences on each array.

The above steps result in the generation of an mRNA expression profile for the initial SPBF that is assayed in the subject methods. The mRNA expression profile generated according to the subject methods provides information concerning the sequence of at least a representative number of the distinct mRNAs in the initial blood fraction, as well as information regarding the quantity or abundance of the distinct mRNAs present in the initial blood fraction. By representative number is meant at least about 10, usually at least about 30 and more usually at least about 50 number % of the total number of distinct mRNAs that may be present in the sample.

Utility

The mRNA expression profiles produced by the subject methods find use in a variety of different applications. Representative applications of interest include, but are not limited to: (a) identification of functional state, e.g., disease, specific markers, including nucleic acid and protein functional state, e.g., disease, specific markers; (b) disease diagnosis and monitoring; etc. Each of these representative specific applications is now discussed separately in greater detail.

Identification of Disease Specific Markers

One application of particular interest is the identification of functional state, such as disease specific, disease status, disease related or other functional state specific, markers, where the markers may be nucleic acids, e.g., RNAs, or the proteins encoded thereby, which markers are found in blood and can be assayed to diagnose the presence or progression of a disease condition. In this application, the mRNA expression profile of an SPBF generated from a subject having a disease of interest, or a representative mRNA expression profile which is the condensation, compilation or average of a plurality of expression profiles generated from a number of individuals suffering from the given disease, e.g., a statistically significant number, is compared to a reference or control expression profile, where this comparison is made to identify mRNAs that are present in different amounts between the two profiles and therefore represent a functional state, e.g., disease, specific marker, e.g., encode a disease specific protein.

The control or reference expression profiles employed in this comparison step are typically profiles that are “normal,” e.g., are profiles generated from subjects not suffering from the given disease of interest. As such, the control or reference expression profiles represent the profile obtained in the absence of the disease of interest. The control profile may be an actual profile that is generated according to the above protocols using an SPBF from a subject that is known to be free of the disease of interest. Alternatively, the control may be a synthetic construct, e.g., a compiled profile that is generated from a number of different individual “normal” profiles. Any convenient control profile may be employed, so long as comparison of the control profile to the mRNA expression profile generated from the subject yields meaningful results in terms of the identification of mRNA species that are present in different amounts in the diseased subject as compared to the control, non-disease subject.

A variety of different control profile generation protocols may be employed to generate the control or reference profile employed in the comparison step. Representative protocols include protocols where the target nucleic acids are generated from a control sample at the same time that the target nucleic acids are generated from the disease sample, and both collections of target nucleic acids are hybridized to either different arrays or the same array, either simultaneously or sequentially, depending on the protocol and the nature of the labels being employed, to generate the reference expression profile. Such reference expression profile generation protocols are further described in U.S. Pat. No. 5,994,076, as well as PCT publication nos. WO 00/22172 and its priority United States patent application, the disclosures of which United States Patent and Patent Application are herein incorporated by reference. Alternatively, a synthetic control set of target nucleic acids may be employed to generate the reference expression profile, where such a protocol is described in PCT publication no. WO 00/65095 and its priority U.S. patent application Ser. No. 09/298,361, the disclosure of which is herein incorporated by reference.

In certain embodiments, the mRNA expression profile generated from the diseased subject is compared to a gene expression database, where the gene expression database is preferably one produced according to the methods described in PCT publication no. WO 00/65095 and its priority U.S. patent application Ser. No. 09/298,361, the disclosure of which is herein incorporated by reference. Of particular interest is a database that incorporates gene-expression profiling data from multiple physiological sources:

1. normal control samples from the healthy individuals, including variation in age, sex, race, etc.

2. normal control samples from healthy individuals under different physiological conditions, like circadian cycles, pregnancy, time of the year and day, amount of physical activity, food, etc.

3. normal control samples from healthy individuals with common disorders, like insomnia, headache, flu infection, cold, exposure to toxic or other compound, like alcohol, drugs, etc.

4. disease samples from disease individuals, without any limitation to the type or kind of disease.

5. disease samples from diseased individuals that are known responders or non-responders to certain therapeutics.

6. samples from individuals or inbred strains with known susceptibility or resistance to disease or other factors.

Preferably, all expression data accumulated in form of the database employed in the comparison step described above is generated using similar technology for RNA purification, target preparation, hybridization, data analysis, etc, such that the data accumulated in the database are homogeneous to each other, such that they can be compared to each other. It is preferred that the gene expression data will be generated not only from SPBF but also from other normal, disease or otherwise (functional state) different tissue, cells, cell and blood fractions, as mentioned above. The main purpose of these additional expression data generated from other physiological sources rather than SPBF is to find a connection or association between discovered differentially expressed genes (in SPBF) and non-SPBF samples. These additional expression data will find use to predict specificity or uniqueness of discovered markers. Comparison expression profiles in disease-specific blood fraction and blood cells/plasma allows one to reveal new markers which can be detected only in SPBF, to the exclusion of other blood fractions/samples.

In identifying the disease specific markers using the subject expression profiles, the above comparison step is employed to identify genes that are differentially expressed in the disease state as compared to the normal, non-disease state, i.e., the reference or control, which differentially expressed genes are then identified as being disease specific or related markers, or at least candidate disease specific or related markers. In identifying disease specific or related markers, of particular interest for the purpose of the invention are genes that are significantly up or down regulated in most cases of particular disease state (markers) in comparison with normal control physiological states, where there is a positive correlation between differences in gene expression level and disease state, for example by measuring Euclidean distance or Pearson's correlation, among others. As such, a substantially consistent, e.g., varying by less than 5%, difference should appear in at least about 30% of a representative number of patients with the disease, preferably at least about 50% of a representative number of patients with the disease and most preferably at least 70% of a representative number of patients with the disease, where representative number typically means at least about 10, usually at least about 50 and more usually at least about 100 or more, e.g., 1000, 2000 or higher. Gene expression level for purposes of identifying differences in expression level is determined in terms of mRNA abundance, where the mRNA abundance is determined relatively or absolutely, as explained above. A difference in expression is viewed as significant in terms of this specification if it is an at least two-fold difference, usually an at least five-fold and more usually an at least ten-fold difference.

Genes that are identified as markers according to the above methods, e.g., as determined through changes in corresponding mRNA abundance level, where the mRNA corresponds to a gene if it is transcribed from that gene, can be used in the discovery of corresponding protein markers. Any known in the art immunological or protein expression analysis technique may be employed to confirm concordance in expression level of an identified nucleic acid marker, e.g., mRNA, and the protein encoded by that mRNA. Examples of such technologies include, but are not limited to, two dimensional gel electrophoresis, mass spectrometry, antibody based technology such as Western blot, ELISA, FACS analysis, etc, where those of skill in the art know how to perform such protocols.

In many embodiments, comparison of expression profiles according to the methods described above simultaneously identifies multiple disease specific/functional state markers, which markers may conveniently be employed together to identify the functional state of the host, e.g., the presence of a disease or other abnormal functional state, such as described in the example below. Thus, the subject methods can be used to simultaneously identify a plurality of disease related or specific markers, e.g., 5, 10, 50, 100, 500 or more. Where multiple disease specific markers are identified, they may conveniently be viewed as a set of disease related specific markers, where specific examples of a set of disease related or specific markers is an mRNA or protein expression profile, which are compendiums of a large number of disease specific markers.

Diagnosis of Disease States

The subject mRNA expression profiles prepared from an SBPF, as described above, can also be employed directly in diagnostic applications. In such applications, an mRNA expression profile is generated from the SPBF of a subject suspected of having a disease of interest. The subject specific mRNA expression profile is then compared to a reference profile that is a profile which is expected to be observed in a subject known, e.g., to have the disease, i.e., a disease specific profile. The subject specific profile can be compared with the reference or disease profile using any convenient protocol, including manual comparison, e.g., visual comparison, and automated comparison, e.g., using a computing comparison means. In many embodiments, a computing means is employed to compare the observed mRNA expression profile with a reference or disease profile.

In this comparison step, the subject specific mRNA expression profile may be compared with a single disease or reference profile, or a plurality of reference profiles each specific for a different disease. For example, the subject specific mRNA expression profile can be compared to a plurality of different disease specific profiles, where by plurality is meant at least 2 and usually at least 5, wherein in many embodiments the number of different profiles with which the subject expression profile is compared may be as high as 10, 50, 100, 500, 1000 or higher. Using this latter embodiment, the subject can be rapidly diagnosed for presence or absence of a large number of diseases using a single subject derived sample.

Monitoring of Disease Progression

The subject mRNA expression profiles also find use in monitoring a host for disease progression, i.e., in tracking the changes in a disease state over time. In these embodiments, mRNA expression profiles are taken from an SPBF obtained from the subject at at least 2 different points during a given time period, e.g., daily, weekly, biweekly, etc., in a 30 day period. The mRNA expression profile obtained at each point during the period is compared to a reference. Changes in the mRNA profile over the given time period are then related to the progression of the disease. In this manner, the disease progression can be monitored to see if it is advancing or retreating. In addition, the affect of a treatment regimen, e.g., one or more pharmacological regimens, can be monitored.

Prognostic and Predictive Diagnostics

In these embodiments, patient subgroups with modified expression of certain genes or gene sets (see e.g., example 5, Table 1b, infra) are followed, retrospectively or prospectively, for the disease outcome or therapeutic effect of a particular drug or therapeutic approach. Correlative analysis of the “expressors” and “non-expressors” with the disease outcome or therapeutic effect allows one to make conclusions on the prognostic and therapy predictive value of the revealed genes or gene sets.

Disease Susceptibility

In these embodiments, subgroups of individuals with a modified expression of certain genes are identified among normal donors and the subgroups are followed up, retrospectively or prospectively, for the susceptibility to certain disease groups (autoimmunity, bacterial infection, viral infection, cancer, etc.) or particular diseases (for example, breast cancer vs. colon cancer). It should be emphasized that the “disease-specific” fraction, in this case, will be comprised of the normal background elements, mainly of blood cell origin, and will reflect important allotypic variations of the immune system that may predetermine individual-specific processes when disease happens.

Alternatively, human individuals or mouse strains with known susceptibility and resistance to certain diseases are tested for the expression profiles of their “disease-specific” fraction to search for the gene profiles correlating with the resistance or susceptibility.

Functional State.

The correlates of various functional states (arousal, depression, natural cycling, etc) can also be searched in the mRNA expression profiles of the “disease specific” fraction. The identification of functional state-related profile variations has both subordinate and independent purposes. The subordinate purpose is to learn to better discriminate disease-related profile elements from others, such as functional state variations. The independent important purpose of learning functional state profiles is related to the association of certain functional states with disease susceptibility (cancer risk of chronic depression) and resistance. The profiling allows one to identify the genes responsible for this susceptibility and resistance.

Applications of Identified Functional State, e.g., Disease Related Markers

The disease related/specific markers, including nucleic acid (e.g., mRNA) and protein markers, identified using the above described protocols find use in a variety of diagnostic and disease management applications. The markers identified using the subject methods are specific for blood or a fraction thereof, e.g., serum, plasma, blood cells/cell subsets, vesicles, etc. As such, the first step in methods of using the identified markers is to obtain the relevant blood fraction. Next, the fraction is assayed for the presence, and often amount, of the relevant marker or markers, where the sample is typically assayed for a plurality of markers, e.g., at least 2, usually at least 5 and more usually at least 10, where the number of different markers for which the sample is assayed may be as great as 50, 100, 500 or more.

There are many different techniques known in the art for identifying the presence of a particular nucleic acid in a sample. For example, RNA markers could be generated by RT-PCR or other technologies based on a combination of one or more of reverse transcription, hybridization and amplification technology, like rolling cycle amplification, ligase chain reaction, transcription-based amplification, amplifiable RNA reporters, etc. In a preferred embodiment, SMART™ cDNA amplification (Clontech Laboratories, Inc., Palo Alto, Calif.) can be used in order to generate amplified cDNA. In other embodiments, amplification of hybridized control targets can be used for generating hybridization target. The amplified products can be detected by well known in art technologies, like gel electrophoresis, quantitative PCR, capillary gel electrophoresis, chromatography, etc. In a preferred embodiment, after the amplification step the product is detected using a nucleic acid array with nucleic acid probe comprising sequences corresponding to the marker RNAs for which the sample is being assayed.

In another embodiment, the sample, e.g., plasma, serum, whole blood or disease-specific particle fraction thereof, e.g., the 4,000×g-20,000×g and often the 4,000×g-10,000×g described above, is assayed for the presence of one or more, typically a plurality, of protein markers, which markers correspond to the identified RNA markers as described above. By plurality is meant at least about 2, usually at least about 5 and more usually at least about 10, where the number may be 50, 100, 500 or more, depending on the particular disease and the number of specific protein markers that have been identified for it. A variety of different protocols may be employed to assay the sample for the presence of the one or more protein markers of interest, where representative assay protocols include, but are not limited to, solid phase immunoassay, FACS analysis, Western blotting, ELISA, and other well known in the art techniques developed for detection specific proteins.

Databases

Also provided are databases of gene expression profiles, where the profiles in the database are profiles prepared according to the subject methods described above. In other words, the databases are collections of disease or functional state specific particular blood fraction gene expression or mRNA profiles. Because the databases of the subject invention are compilations or collections of gene expression profiles prepared as described above, the subject databases have a number of advantages, where such advantages include, but are not limited to: the generation of more compact information (number/versus image file); the identification of expression levels that are not dependent on type of array, hybridization conditions, lot of array, etc. These advantages are significant in that expression data obtained with the subject methods does not need annotation to be meaningful; and the database generated from the data can be universal, i.e., it can be generated using data generated in different labs, or at different times, or even using different types of arrays.

The subject expression profiles and databases thereof may be provided in a variety of media to facilitate their use. “Media” refers to a manufacture that contains the expression profile information of the present invention. The databases of the present invention can be recorded on computer readable media, e.g. any medium that can be read and accessed directly by a computer. Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. One of skill in the art can readily appreciate how any of the presently known computer readable mediums can be used to create a manufacture comprising a recording of the present database information. “Recorded” refers to a process for storing information on computer readable medium, using any such methods as known in the art. Any convenient data storage structure may be chosen, based on the means used to access the stored information. A variety of data processor programs and formats can be used for storage, e.g. word processing text file, database format, etc.

As used herein, “a computer-based system” refers to the hardware means, software means, and data storage means used to analyze the information of the present invention. The minimum hardware of the computer-based systems of the present invention comprises a central processing unit (CPU), input means, output means, and data storage means. A skilled artisan can readily appreciate that any one of the currently available computer-based system are suitable for use in the present invention. The data storage means may comprise any manufacture comprising a recording of the present information as described above, or a memory access means that can access such a manufacture.

A variety of structural formats for the input and output means can be used to input and output the information in the computer-based systems of the present invention. One format for an output means ranks unknown disease profiles possessing varying degrees of similarity to a reference known disease profile. Such presentation provides a skilled artisan with a ranking of similarities and identifies the degree of similarity contained in the test unknown disease profile.

The subject expression profile databases find use in a number of different applications. For example, where one has an expression profile of interest, one can search the database to determine whether that profile is present in the database and, if so, readily identify the source of the expression profile, i.e., the identify of the sample that has the given expression profile.

The comparison of an expression profile obtained from an assayed sample and expression profiles present in the database, i.e. reference expression profiles, is accomplished by any suitable deduction protocol, Al system, statistical comparison, etc. Methods of searching databases are known in the art. See, for example, U.S. Pat. No. 5,060,143, which discloses a highly efficient string search algorithm and circuit, utilizing candidate data parallel, target data serial comparisons with an early mismatch detection mechanism. For other examples, see U.S. Pat. No. 5,720,009 and U.S. Pat. No. 5,752,019, the disclosures of which are herein incorporated by reference.

Preferably, the subject databases will incorporate gene-expression profiling data from multiple physiological sources, which sources include:

1. normal control samples from the healthy individuals, including variation in age, sex, race, etc.

2. normal control samples from healthy individuals under different physiological conditions, like circadian cycles, pregnancy, time of the year and day, amount of physical activity, food, etc.

3. normal control samples from healthy individuals with common disorders, like insomnia, headache, flu infection, cold, exposure to toxic or other compound, like alcohol, drugs, etc.

4. disease samples from disease individuals, without any limitation to the type or kind of disease.

5. disease samples from disease individuals that are known responders or non-responders to certain therapeutics.

6. samples from individuals or inbred strains with known susceptibility or resistance to disease or other factors.

Preferably, all expression data accumulated in form of the database is data that is generated using similar technology for RNA purification, target preparation, hybridization, data analysis, etc. Such uniformity in data preparation provides for a homogeneous database in which the individual data points can be compared to each other. Preferably, the gene expression data will be generated not only from disease-specific particular blood fraction described in greater details above but also from other normal and disease tissues, cells, cell and blood fractions. The main purpose of these additional expression data generated from other physiological sources rather than disease-specific particular blood fraction is to find connections or associations between discovered differentially expressed genes (in disease specific blood fraction) and disease states of disease associated tissues. These additional expression data will find use in predicting specificity or uniqueness of discovered markers. Comparison of expression profiles in SPBF and blood cells/plasma allows one to reveal new markers which can be detected only in SPBF as apposed to other blood fractions, and finds utility as described above.

Kits

Also provided are kits for use in practicing the subject invention. The subject kits typically include a means for generating an expression profile from an SPBF, whole blood or other acceullar or cellular blood fraction. In one embodiment, such means generally include one or more reagents for generating the target nucleic acids from the disease specific particular blood fraction, including, but not limited to: enzymes (polymerases, reverse transcriptases, etc); nucleotides, including labeled nucleotides; primers, including labeled primers; buffers, and the like. The kits may also include arrays for use in generating the subject expression profile arrays, such as the arrays described above. In addition to the above means for generating the mRNA or expression profiles, the subject kits may also include one or more reference profiles, including a database of expression profiles as described above, as well as a means for accessing such a reference profile(s) remotely, e.g., a URL address. The reference profiles can be control or normal profiles, e.g., for identifying novel disease specific markers, or known disease profiles, e.g., in diagnostic and disease monitoring applications.

In yet other embodiments, the kits are kits for use in obtaining a protein profile of whole blood or blood fraction and making a diagnosis based thereon. In these embodiments, the kits typically include a combination, e.g., an array, of a plurality of specific binding pair members that are specific for disease markers, particularly protein disease markers, and more preferably protein disease markers that are endogenous human proteins. The subject arrays generally include at least 5 different specific binding pair members, usually at least 10 different specific binding pair members and more usually at least 20 different specific binding pair members, where each of these different binding pair members specifically binds to a different disease specific protein marker. In addition, the kits of this embodiment also generally include one or more reference protein profiles, or means for accessing such from a remote location, e.g., a URL address.

The kits may also include a means for obtaining and/or storing a blood sample and reagents for isolation of SPBF or other blood fraction, e.g., syringes, vacutainers, test tubes, buffers, nucleic acid or protein isolation reagents, etc.

Also present in many embodiments of the subject kits are instructions for practicing methods of producing the subject expression profiles, e.g., nucleic acid and protein profiles, and/or using the profiles in identification of disease markers or diagnosis/disease monitoring applications, where these instructions may be present on one or more of: a package insert, the packaging, reagent containers and the like.

Advantages Provided by the Subject Invention

Use of SPBF to obtain mRNA expression profiles, as well as the markers identified therewith, provides a number of distinct advantages. The advantages are based on the nature of the SPBF, which is comprised predominantly of vesicles released in the blood by diseased or activated organ/tissue/cells (or other cells of the organism activated or injured in related to the disease).

As such, expression profiles and diagnostic markers obtained therefrom, as described above, are clinically applicable to the early diagnosis of disease states and can also be used as preventive medical diagnostic tools to treat diseases before visual symptoms appear. As such, the subject invention provides for the diagnosis of disease states at early stages in order to identify a disease state, its stage, particular subclass, patient-specific variations, etc. The subject invention also allows one to rationally predict therapies, like biotherapy, chemotherapy, radiotherapy, etc., for treatment of particular disease state. In addition, the subject invention can be used to provide an estimation of effectiveness of therapy and a prediction of alternative therapy. Furthermore, the markers identified by the subject methods can be used to develop drugs, which can be used for treatment of particular disease states.

As such, the subject invention provides for a number of significant advantages and features, which make it a significant contribution to the art of disease diagnostics.

The following examples are offered by way of illustration and not by way of limitation.

### EXPERIMENTAL

### EXAMPLE 1

### Preparation of SPBF

A. Isolation of Disease-Specific Particle Fraction from Blood.

The following protocol describes the purification of SPBF from 1-10 ml of whole blood. The conditions described in the protocol were used for purification of 4,000 to 20,000×g disease-specific particle fraction (as a precipitate at stage 7). The 300 to 4,000×g fraction was collected as a precipitate after stage 6. The 20,000 to 100,000×g fraction was collected as a precipitate after an additional centrifugation step of the supernatant generated at step 7 at 100,000×g at 4° C. for 1 hr in TL100 (Beckman) centrifuge.

Equipment:

Beckman TJ-6 Table top Centrifuge

Eppendorf Centrifuge with refrigerator 5417.

Isolation of Disease-Specific Particulate Plasma Fraction.

1. Collect blood into yellow top vacutainers (Beckton Dickenson) tubes.

2. Keep no more than 24 h (room temperature or 4° C.).

3. Centrifuge at 300×g (1200 rpm in Beckman TJ-6 centrifuge) for 15 min (room temperature), collect supernatant in tubes that fit into a microcentrifuge (1.5 ml Eppendorf or 2 ml screw caps).

4. Freeze and keep plasma at −70° C. If option to isolate RNA immediately is available, go to step 6 without freezing/thawing step.

5. Thawing Plasma: Place test tubes with 1 ml of frozen plasma in a shallow dish of water to thaw gradually. Gently vortex occasionally to mix plasma.

6. Transfer 1 ml of plasma into Eppendorf 1.5 ml test tube. Centrifuge plasma at 4000×g (about 6100 rpm in Eppendorf 5417) for 30 min. at 4° C., collect supernatant into another 1.5 ml test tube.

7. Centrifuge supernatant at 20,000×g (14,000 rpm in 5417 Eppendorf Centrifuge) for 30 min at 4° C.

Note: Pellet is often not visible. Use the tube position in the rotor to identify the suspected region of the pellet and do not disturb this area while removing supernatant. Also, with a tilted rotor, the pellet can slip down to the bottom of the tube so try not to disturb this area either. For best results, remove supernatant immediately after centrifugation.

B. Isolation of Disease-Specific RNA from Plasma

The following protocol describes the procedure for purification of disease-specific total RNA from 1-10 ml of blood samples. It should be noted that if different equipment, reagents, or blood volume are used, it is necessary to optimize protocol to these changes. Some parameters, like exact temperature for incubation, time of storage, g-forces, reagents choice, etc. could be changed without significant changes in total RNA performance.

Reagents and Equipment:

Nucleospin RNA 2 Kit (CLONTECH cat. #K3064-2)

β-Mercapto Ethanol

Linear Acrylamide (Ambion, 5 μg/μl)

SUPERase●In™ RNase inhibitor, 20μ/μl (Ambion, Inc. cat. #2696).

DNase I (RNase-free)—1μ/μl (Epicentre, Cat. K99058K)+10×buffer

MHC amplimer PCR primer set (CLONTECH, Cat. 9223)

SYBR Green dye (Molecular Probes)

RNA Purification

The following protocol is for 1.0 ml starting plasma volume or bigger volume (up to 10 ml).

1. For each plasma sample pellet in Eppendorf test tube add mixture of: 300 μl of RA1 buffer (room temperature), 3 μl β-Mercapto Ethanol and 2 μl of linear acrylamide (5 mg/ml).

2. Gently pipet up and down with P1000, rinsing the side or bottom where the pellet is expected to be.

3. Add ¾ volume (225 μl) of 100% Ethanol, mix well.

4. Load sample from step 3 onto NucleoSpin RNA II column.

5. Spin at 8000 rpm for 2 min.

6. Add 600 μl of Buffer RA3 to NucleoSpin column. Centrifuge at 8,000 rpm for 30 sec. Place the NucleoSpin column into a clean tube.

7. Repeat washing step 6 two more times.

8. Place column in a clean tube and spin at 14,000 rpm for 1 min. to completely remove wash buffer.

9. Transfer to an RNase free 1.5 ml Eppendorf tube. Add 50 μl of RNase free water directly to filter (do not close cap). Allow filter to soak 2 min. Close cap and elute by centrifuging 14,000 rpm for 1 min.

10. Repeat step 11 for a secondary elution, collect eluate in the same tube. Total volume of RNA sample is 100 μl. (Take 10 μl aliquot from each test tube and test genomic DNA impurities using MHC primers and 1 ng of genomic DNA as a calibration standard).

11. To 90 μl of RNA sample from step 10 add mixture of (10 μl of 10×DNase I buffer, 5 μl of Superase-In, 2 μl of linear acrylamide and 2 μl of DNase (1 uμl)). Incubate 30 min at 37° C.

12. Add to each RNA sample 400 μl of RA1 buffer, then three fourths volume (375 μl) of 100% ethanol, mix well.

13. Load samples 400 μl from step 12 onto Nucleospin column. Centrifuge at 8,000 rpm for 30 sec. Repeat loading and centrifugation for the rest of the sample.

14. Repeat steps 6-10. Elute RNA in total 50 μl of water, use 35 μl and 20 μl for first and second elution respectively.

15. Measure RNA concentration by RT-PCR using housekeeping genes (MHC cDNA) or SYBR Green dye and human total peripheral leukocyte RNA as a standard. Observed yields should be about 1-5 ng per ml of plasma.

16. Store RNA frozen at −70° C.

### EXAMPLE 2

### Generation of Hybridization Probe from Disease Specific Plasma Fraction and Expression Profiling with Atlas Human 1.2 Expression Arrays

The protocol below describes a variation of an expression profiling experiment conducted on SPBF (4,000×g-20,000×g) isolated from 1 ml of human blood as described in example 1 and 2, above. The protocol should be considered as an illustration. Some modification in conditions, reagents, equipments, etc. are possible and rather obvious for the person skilled in the art.

Part A. First-Strand cDNA Synthesis

A. Reagents and Equipment:

SMART PCR cDNA synthesis kit (CLONTECH, Cat. K1052)

Advantage cDNA PCR kit (CLONTECH, Cat. K1905)

Atlas Human 1.2 Array (CLONTECH, Cat. #7850)

AtlasImage Software (CLONTECH, Cat. #V1211)

Atlas Navigator Software (CLONTECH, Cat. #1220)

Nucleospin PCR Extraction kit (CLONTECH, Cat.K3051)

Linear Acrylamide (Ambion, 5 μg/ul)

SUPERase●In™ RNase inhibitor, 20u/ul (Ambion, Inc. Cat. #2696).

Klenov enzyme (2μ/μl)+10×buffer (Roche Molecular Biochemicals, Cat. #1008404)

a-33P dATP, 10 μCi/μl, 2500 Ci/mmol (Amersham)

Eppendorf Centrifuge with refrigerator 5417

Thermal Cycler (MJ Research, PTC-200 model).

Phosphorimager (Molecular Dynamics, Storm 600)

All reagents and protocol from SMART PCR cDNA synthesis kit

B. Protocol

B.1 cDNA Synthesis

1. Combine the following reagents in a 0.5-ml microcentrifuge tube:

| | |
|---|---|
| RNA sample (1-5 ng) | 50 μl |
| cDNA Synthesis (CDS) primer (12 μM) | 7 μl |
| SMART II Oligonucleotide (12 μM) | 7 μl |
| Total volume | 64 μl |

2. Incubate the tube at 65° C. in a thermal cycle for 2 min.

3. During the annealing time, prepare a Master Mix in a separate tube. (Do not add RT enzyme until immediately before adding mix to sample, in step 6):

| Master Mix | |
|---|---|
| 5× First-Strand Buffer | 20 μl |
| DTT (100 mM) | 2 μl |
| dNTP mix (10 mM) | 10 μl |
| Superasin (20 x) | 5 μl |
| Power Script | 5 μl |
| Total volume | 42 μl (per reaction) |

Mix well by pipetting

4. Change temperature in PCR machine to 42° C. Incubate tubes at 42° C. for 2 min.

5. Add 42 μl Master Mix to the tube (from step 2). Mix well by pipetting.

6. Incubate the tubes at 42° C. for 30 min. Purify by Nucleospin PCR filter (if you need to stop you can store at 4° C. up to one day, or −20° C. if longer.

B.2 Purify cDNA by NucleoSpin PCR Purification Kit

1. Add 400 μl NT2 Buffer to the sample (from step 6). Mix well.

2. Place Nucleospin PCR Filter into a collection tube, then pipet sample onto filter.

3. Centrifuge at 14,000 rpm (Eppendorf centrifuge), 1 min. Discard collection tube and transfer filter to a fresh tube.

4. Add 700 μl NT3 Buffer to filter.

5. Centrifuge at 14,000 rpm, 1 min. Discard collection tube and transfer filter to a fresh tube.

6. Repeat steps 5-6 twice.

7. Transfer to a new collection tube and spin at 14,000 rpm for 1 minute to dry filter.

8. Transfer filter to a fresh 1.5-ml tube.

9. Elute first-strand cDNA by adding 55 ul Milli Q water to the filter. Incubate 2 minutes with lid open. Close lid and centrifuge at 14,000×g for 1 minute. Elute a second time into the same tube using 30 μl water. Total elution volume equals about 80-85 μl.

Part B. SMART cDNA Amplification by LD-PCR

9. Preheat the PCR thermal cycler to 95° C.

10. Place 79 μl of First-Strand cDNA from step 7 (Part A) into a 0.5-ml PCR tube.

11. Prepare a master mix in a separate tube.

| Master Mix | |
|---|---|
| 10× Advantage PCR buffer | 10 μl |
| Milli Q Water | 5 μl |
| 5′ PCR primer | 2 μl |
| 50× dNTP mix | 2 μl |
| 50× Advantage Polymerase Mix | 2 μl |
| Total volume | 21 μl (per reaction) |

12. Add 21 μl of the Master Mix to EDNA sample (step 10). Mix well by pipetting.

13. Place tubes in a preheated (95° C.) thermal cycler.

14. Commence thermal cycling using the following program:

| | | | | |
|---|---|---|---|---|
| Step 1. | 95° C. | | 1 min. | |
| Step 2. | 95° C. | | 15 sec. | |
| Step 3 | 65° C. | {open oversize bracket} | 30 sec. | x cycles |
| Step 4 | 68° C. | | 3 min. | |
| Step 5 | 4° C. maintain | | | |

15. For each PCR tube, determine the optimal number of PCR cycles:

a. Visualize 5 μl from the 21-cycle PCR alongside Amplisize molecular weight marker (BioRad) on a 1.2% Agarose gel/1×TAE run at 2V/cm for 1.5 hours. If needed, run three additional cycles (steps 2 to 5 above equals to 1 cycle) with the remaining 95 μl of the PCR mixture.

b. Repeat step (a.) above until a sample begins to amplify. Depending on the intensity, add not more than three cycles to this sample. Use this sample as a calibration standard. Add cycles to the other samples until their intensities become roughly the same as the standard. Once each sample has been optimally cycled store them at 4° C. up to 1 day, −20° C. if longer.

16. When the cycling is completed, adjust the reaction volume to 100 μl with TE, pH 7.5.

17. Add 400 μl NT2 Buffer (Nucleospin PCR purification kit) to the sample. Mix well.

18. Place Nucleospin Filter into a collection tube, then pipet sample onto filter.

19. Centrifuge at 14,000 rpm for 1 min. Discard collection tube and transfer filter to a fresh tube.

20. Add 700 μl NT3 Buffer to filter.

21. Centrifuge at 14,000 rpm for 1 min. Discard collection tube and transfer filter to a fresh tube.

22. Repeat steps 5-6 twice.

23. Transfer to a new collection tube and spin at 14,000 rpm for 1 minute to dry filter.

24. Transfer filter to a fresh 1.5-ml tube.

25. Add 50 μl NE Elution Buffer to filter, do not close lid.

26. Allow filter to soak for 2 min.

27. Close lid and centrifuge at 14,000 rpm for 1 min to elute PCR product.

28. Repeat steps 25-27 one time, then discard filter.

29. Analyze a 5 μl sample of each PCR product alongside Amplisize markers (BioRad) on a 1.2% agarose/EtBr gel in 1×TAE buffer.

30. Quantitate purified PCR product using UV Spectrophotometer.

Part C. Generation ³³P-labeled Hybridization Probe by Primer Extension.

1. Probe can be synthesized with up to 500 ng of purified PCR product (step 29. Part B). Assemble the probe reaction in PCR test tube as follows:

| SMART PCR product (up to 33 ul) | X | μl |
|---|---|---|
| Nuclease free H₂O (Bring volume up to 33 ul) | 33-X | μl |
| 10× CDS primer (membrane specific) | 1 | μl |
| | 34 | μl total |

2. In PCR thermocycler heat test tube at 96° C. for 2 minutes to denature the template, then incubate at 50° C. for 2 minutes.

3. Meanwhile, assemble master mix. For each reaction add

| | | |
|---|---|---|
| 10× Klenow Buffer | 5 | μl |
| dCTP, dGTP, dTTP (0.5 mM each) | 5 | μl |
| ³³-P a-dATP | 5 | μl |
| Klenow | 1 | μl |
| | 16 | μl total |

4. Without removing the tube from thermocycler, add 16 μl of the master mix to each sample. Mix well by pipetting.

5. Incubate at 50° C. for 30 minutes. Add 2 μl of 0.5M EDTA to stop the reaction.

6. Purify probe by Nucleospin PCR purification kit.

a. Add 350 μl NT2 buffer to sample. Mix well. Apply to a Nucleospin column/elution tube and spin at 14,000 rpm for 1 min.

b. Transfer to a new elution tube and wash column with 350 μl of NT3 Wash buffer (note* be sure to add required amount of ethanol to NT3 before first use). Spin at 14,000 rpm for 1 min. Repeat NT3 wash twice more.

c. Place column in a clean 1.5 ml microcentrifuge tube. Open column and apply 100 μl NE buffer. Leave column lid open (closing lid will force NE out) and allow column to soak for 2 minutes. Spin at 14,000 rpm for 1 min.

d. Count probe in a scintillation counter. Observed counts have been between 6,000,000 and 30,000,000 DPM.

Part D. Atlas Array Pre-Hybridization/Hybridization

1. Prepare hybridization solution for each membrane:

a. Prewarm ExpressHyb™ Hybridization Buffer (Clontech, Palo Alto, Calif.) at 68° C.

b. Combine 50 μl of 20×SSC and 50 μl of Blocking Solution. Mix well.

c. Boil for 5 min, then quickly cool on ice for 2 min.

d. Combine with 5 ml prewarmed ExpressHyb hybridization buffer an keep at 68° C. until use.

2. Fill hybridization bottles with dH₂O.

3. Wet the membrane with dH₂O and shake off excess. Place the membrane into a hybridization bottle.

4. Pour off dH₂O, then add the solution prepared in step 1.

5. Pre-hybridize for 60 min with continuous agitation at 68° C.

Hybridization

1. Mix 50 μl of 20×SSC, 50 μl of Blocking Solution, and your purified probe.

2. Boil for 5 min, then chill on ice 2 min.

3. Add probe to hybridization solution.

4. Hybridize while rotating at 5 to 7 rpm in roller bottle hybridization incubator overnight.

Washes

1) Prepare wash solutions the night before. Each small bottle will require 450 ml of Wash buffer 1 and 150 ml of Wash buffer 2.

High Salt, Wash buffer 1-2×SSC, 1% SDS (1 liter):

a) Shake 20×SSC stock solution to mix; add 100 ml to 1L bottle.

b) Add 850 ml milli-Q water.

c) Add 50 ml of 20% SDS.

d) Shake well and incubate in 68° C. oven.

Low Salt, Wash buffer²-0.1×SSC, 0.5% SDS (1 liter):

a) Shake 20×SSC stock solution to mix; add 5 ml to 1 L bottle.

b) Add 970 ml milli-Q water.

c) Add 25 ml of 20% SDS.

d) Shake well and incubate in 68° C. oven.

Note* Make sure all buffers are prewarmed at 68° C. Set up radioactive liquid waste receptacle.

2) Pour Wash buffer 1 into a plastic beaker (w/pouring spout).

3) Remove first bottle from oven. Close oven.

4) Quickly remove cap and discard probe hybridization solution into waste beaker.

5) Place bottle on rack, then QUICKLY pour 10-20 ml of Wash buffer 1 into bottle.

*This step must be performed quickly to prevent non-specific background from drying to the membrane.

6) Quickly close bottle, then rock bottle back and forth to rinse off excess hybridization solution.

7) Remove cap and discard rinse into waste beaker.

8) Quickly pour Wash buffer 1 into the bottle until it will be ˜80% full.

9) Close bottle, then shake until membrane is released from side of bottle.

10) Shake bottle a few more times for an even wash.

11) Allow membrane to re-attach to side of bottle and return bottle to oven.

12) Repeat steps 3-11 for remaining bottles.

13) Increase rotation to max speed (15 rpm).

14) Make sure all membranes are attached to side of bottle.

a) If not, hold bottle upright or upside-down until membrane reattaches.

b) Try reversing the position of the bottle in the oven (i.e. cap on right side vs. cap on left side).

c) If nothing else works, shake bottle vigorously a few more times, hold upright, then return to oven.

15) Wash membranes for 30 minutes; try not to exceed 40 minutes.

16) Remove first bottle from oven, then repeat steps 7-12. Repeat for remaining bottles.

17) Wash membranes for 30 minutes; try not to exceed 40 minutes.

18) Remove first bottle from oven, then repeat steps 7-12. Repeat for remaining bottles.

19) Wash membranes for 30 minutes; try not to exceed 60 minutes.

20) Remove first bottle from oven, then repeat steps 7-12, using Wash buffer 2. Repeat for remaining bottles.

21)Wash membranes for 30 minutes; DO NOT EXCEED 30 MINUTES IN WASH BUFFER 2.

22) Remove all bottles from oven, place on rack.

23) Make sure all membranes are completely submerged in the wash buffer. Shake bottles if necessary.

24) Quickly dip membrane in milli-Q water. Place on Whatman 3M blotting paper to dry. Dry completely and cover with 1.5 micron thick mylar before exposing to 33-P low energy phosphoimager cassette.

Part E. Exposure and Data Analysis

Recommended: overnight exposure on Molecular Dynamics low energy screen for a short exposure and 7 to 14 days for long exposure. Scan short exposure at 200 micron and long exposure at 100 micron resolution. Use AtlasImage™ imaging software (Clontech, Palo Alto, Calif.) to convert *.gel file to aligned *.gmd files. AtlasImage can be used to make comparisons between one control and one experimental array or to generate normalization coefficients using the global sum normalization method. AtlasImage can also be used to generate data reports that can be used in conjunction with AtlasNavigator™ processing software (Clontech, Palo Alto Calif.) to make larger group comparisons. Using AtlasImage together with AtlasNavigator makes it possible to compare groups or individual arrays to obtain differential gene expression data.

### EXAMPLE 3

### Expression Profiling of Various Blood Fractions

Using the protocols described above in Examples 1 and 2, the following blood fractions obtained from a healthy donor were analyzed to generate expression profiles:

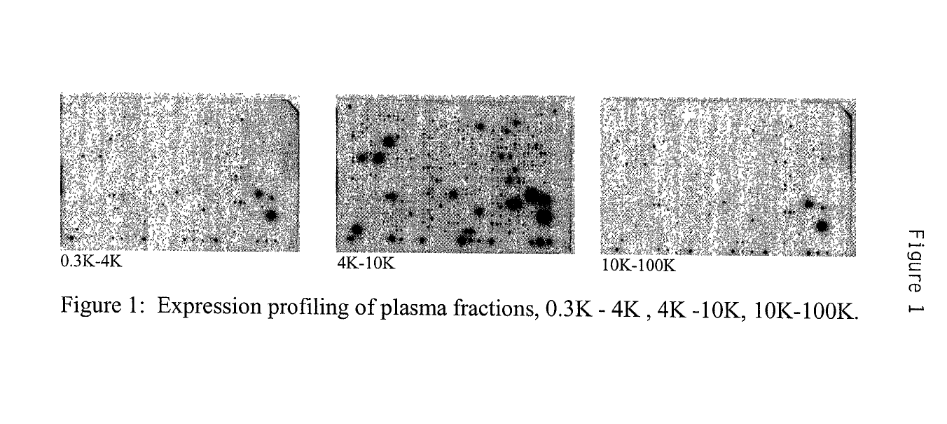

(a) 0.3-4,000×g fraction; (b) 4,000-10,000×g fraction (SPBF); and (c) 10,000-100,000×g fraction. The results are provided in FIG. 1 and clearly demonstrate that mRNA is present in the disease specific particular blood fraction (i.e. the 4,000-10,000×g), but is present in too small of an amount in the other two other fractions (0.3-4,000×g and 10,000-100,000×g) to be useful for expression profiling generation with array based technology.

### EXAMPLE 4

### Comparison of Expression Profiles in “Normal” and Myeloma Disease-Specific Plasma Fraction

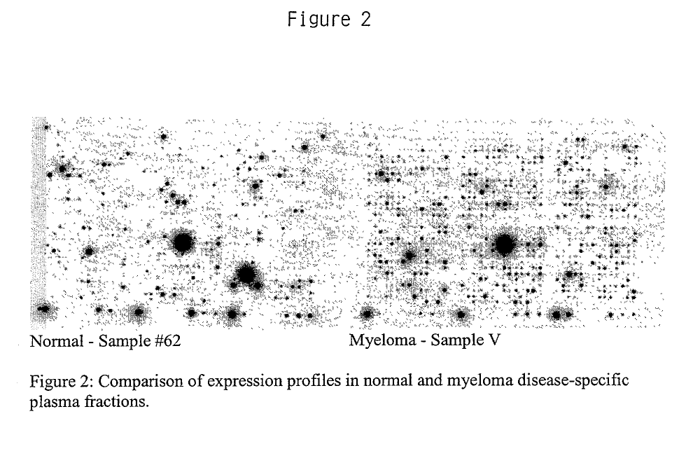

RNAs from disease specific particular blood fraction (4,000-20,000×g) of normal donor and myeloma patients were purified, converted to hybridization probes and hybridized with Atlas Human 1.2 Expression Arrays, according to Examples 1, 2 and 3 above. FIG. 2 provides the the Expression Profiles generated from the disease and normal samples. The results clearly demonstrate significant differences in the mRNA composition of normal and disease (myeloma) samples.

### EXAMPLE 5

### Comparison of Expression Profiles in Disease-Specific Plasma Fraction from Normal Donors and Chronic Fatigue Syndrome Patients

RNAs from SPBF (4,000-20,000×g) of normal donor and CFS patients were purified, converted to hybridization probes and hybridized with Atlas Human 1.2 Expression Arrays, according to Examples 1, 2 and 3 above. The results clearly demonstrate significant differences in the mRNA composition of samples from normal donors and chronic fatigue syndrome (CFS) patients.

Genes that are differentially expressed in CFS patients vs. normal donors are presented in Table 1a as genes that are overexpressed or downmodulated in more, than 66% CFS patients. A gene was considered overexpressed (red background) if the corresponding AtlasImage figure exceeded the average for this gene in normal donors more than 3 fold. A gene was considered downmodulated (blue background) if the corresponding AtlasImage figure for this gene was more than 10 times less than normal donors' average for this gene. Of the 5 genes shown in table 1a each of the CFS patient has a modified expression of at least 2 genes. Thus the set contains good candidates markers to reliably identify the disease state in general and CFS pathology in particular.

Genes that are differentially expressed in different CFS patients (Table 1b) allow to subdivide the patients into subgroups that may have different prognosis or may need different therapeutic approaches. The genes of these sets are good candidates for correlative analysis with clinical outcome and/or therapeutic response to different therapeutic agents or strategies. Moreover, the analyses of genes overexpressed or downmodulated in a particular subgroup provides a clue for an adequate therapeutic strategy. Thus, one of the genes overexpressed in CFS patients C and G is TNF receptor encoding gene. No other CFS patients overexpress the gene. Since TNF is one of the major inflammatory cytokines, the overexpression of its receptor somewhere in the organism may be a serious pathogenic factor. Thus, a therapeutic approach using TNF blockade by, for example, some existing drug, such as Embrel (by Immunex, Inc.) is worth trying in this particular subgroup of CFS patients.

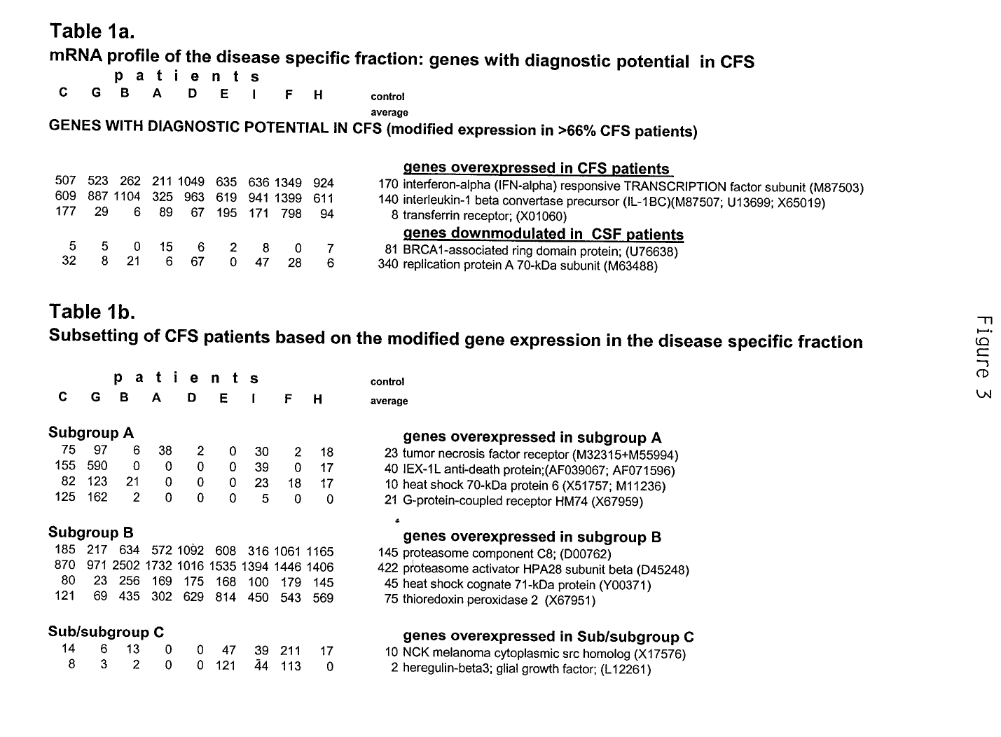

Tables 1a and 1b are provided in FIG. 3.

### EXAMPLE 6

### A Pathway from Expression Profiling to Diagnostic Markers that can be Screened with Proteomic Techniques Traditional for Diagnostic Labs

Sets of genes with modulated expression in disease provide candidates for the search of markers that can be further used for diagnostic purposes in conjunction with traditional proteomic techniques. The array-revealed overexpression of the TNF receptor gene in disease-specific fraction of a subpopulation of CFS patients and the identification of this subpopulation as a target for anti-TNF receptor therapy puts forward a task of the identification of this subpopulation by traditional techniques used in diagnostic labs.

Flow cytometry search for TNF receptor using commercial fluorochrom-labeled anti-TNFR antibody is performed using multicolor staining of blood cells with blood cell differentiating antibodies (anti-CD19, anti-CD3, anti-CD4, anti-CD8, anti-CD14, anti-CD16) that allow to identify blood cell subsets with maximal modification of the expression of surface TNF receptor. All the details of multicolor surface staining of blood cells for FACS analysis are well known for those skilled in the art.

ELISA test for soluble TNF receptor is performed using one anti-TNF receptor monoclonal antibody as a plastic-attached capturing substrate and the second one, chromogen-labeled, as a developing factor to test if any TNF receptor molecules were captured by the first antibody from patient's plasma or serum sample. All the details of this sandwich procedure are well known for those experienced in the art.

It is evident from the above results and discussion that the present invention allows one to substantially accelerate the search for disease specific markers by combinational usage of a specific blood fraction enriched with disease related elements and highthroughput array technology. Contrary to other approaches currently available, the strategy of the present invention is not limited to a particular disease and allows one to simultaneously look for two different groups of markers, specifically, (1) pathology-related markers, and (2) markers showing patient-specific variation in expression of such markers. Markers of the first group are important in all three diagnostic aspects (disease diagnostics, prognosis, and prediction of appropriate individual therapy). Markers of the second group are most important for predictive therapy. Various combinations of multiple markers belonging to both groups may be further used to create disease-specific or universal diagnosticums. As such, the subject invention represents a significant contribution to the art.

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention. Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

What is claimed is:

1. A method of generating an mRNA expression profile, said method comprising:
(a) providing an acellular mRNA containing blood fraction that contains a plurality of distinct mRNAs;
(b) generating a plurality of distinct target nucleic acids from said acellular mRNA containing blood fraction;
(c) contacting said plurality of distinct target nucleic acids with an array of immobilized probe nucleic acids under hybridization conditions such that complementary target and probe nucleic acids form duplex structures immobilized on the surface of said array; and
(d) detecting any resultant duplex structures to obtain said expression profile.

2. The method according to claim 1, wherein said acellular mRNA blood sample is isolated from blood by differential centrifugation between about 2,000 and 20,000×g.

3. The method according to claim 2, wherein said acellular mRNA blood sample is isolated from blood by differential centrifugation between about 4,000 and 10,000×g.

4. The method according to claim 1, wherein said generating step employs a template dependent primer extension reaction.

5. The method according to claim 4, wherein said template dependent primer extension reaction employs gene specific primers.

6. The method according to claim 4, wherein said template dependent primer extension reaction employs random primers.

7. The method according to claim 4, wherein said template dependent primer extension reaction employs oligo dT primers.

8. The method according to claim 4, wherein said template dependent primer extension reaction comprises an amplification step.

9. The method according to claim 1, wherein said nucleic acid targets are labeled prior to said hybridization step.

10. The method according to claim 9, wherein said nucleic acid targets are labeled with a directly detectable label.

11. The method according to claim 9, wherein said nucleic acid targets are labeled with an indirectly detectable label.

12. A method of identifying a marker(s) related to a disease condition, said method comprising:
(a) generating a disease mRNA expression profile using the method of claim 1 from a sample obtained from a subject having said disease;
(b) comparing said disease mRNA expression profile to a control mRNA expression profile to detect any mRNAs that are present at a level in said disease profile that is different from their level in said control profile; and
(c) identifying at least one of said detected mRNAs that is present at a level in said disease profile that is different from its level in said control profile as a marker specific for said disease.

13. The method according to claim 12, wherein said method further comprises generating said normal mRNA expression profile.

14. The method according to claim 12, wherein said acellular mRNA blood sample is isolated from blood by differential centrifugation between about 2,000 and 20,000×g.

15. The method according to claim 13, wherein said acellular mRNA blood sample is isolated from blood by differential centrifugation between about 4,000 and 10,000×g.

16. The method according to claim 12, wherein said generating step employs a template dependent primer extension reaction.

17. The method according to claim 16, wherein said template dependent primer extension reaction employs gene specific primers.

18. The method according to claim 16, wherein said template dependent primer extension reaction employs random primers.

19. The method according to claim 16, wherein said template dependent primer extension reaction employs oligo dT primers.

20. The method according to claim 16, wherein said template dependent primer extension reaction comprises an amplification step.

21. The method according to claim 12, wherein said nucleic acid targets are labeled prior to said hybridization step.

22. The method according to claim 21 wherein said nucleic acid targets are labeled with a directly detectable label.

23. The method according to claim 21, wherein said nucleic acid targets are labeled with an indirectly detectable label.

24. The method according to claim 12, wherein at least two different disease specific markers are identified.

25. A method of determining whether a subject has a disease state, said method comprising:
(a) generating an mRNA expression profile by:
(i) obtaining an acellular mRNA containing blood fraction that contains a plurality of distinct mRNAs from said subject;
(ii) generating a plurality of distinct target nucleic acids from said acellular mRNA containing blood fraction;
(iii) contacting said plurality of distinct target nucleic acids with an array of immobilized probe nucleic acids under hybridization conditions such that complementary target and probe nucleic acids form duplex structures immobilized on the surface of said array; and
(iv) detecting any resultant duplex structures to obtain said expression profile;
(b) using said expression profile to determine whether said subject has said disease state.

26. The method according to claim 25, wherein said determining step (b) comprises comparing said expression profile to a reference.

27. The method according to claim 26, wherein said reference is an expression profile.

28. The method according to claim 25, wherein said acellular mRNA blood sample is isolated from blood by differential centrifugation between about 2,000 and 20,000×g.

29. The method according to claim 28, wherein said acellular mRNA blood sample is isolated from blood by differential centrifugation between about 4,000 and 10,000×g.

30. The method according to claim 25, wherein said generating step employs a template dependent primer extension reaction.

31. The method according to claim 30, wherein said template dependent primer extension reaction employs gene specific primers.

32. The method according to claim 30, wherein said template dependent primer extension reaction employs random primers.

33. The method according to claim 30, wherein said template dependent primer extension reaction employs oligo dT primers.

34. The method according to claim 30, wherein said template dependent primer extension reaction comprises an amplification step.

35. The method according to claim 25, wherein said nucleic acid targets are labeled prior to said hybridization step.

36. The method according to claim 3 5, wherein said nucleic acid targets are labeled with a directly detectable label.

37. The method according to claim 35, wherein said nucleic acid targets are labeled with an indirectly detectable label.

38. A method of determining whether a subject has a disease condition, said method comprising:
(a) obtaining a blood sample from said subject;
(b) quantitating the amount of a plurality of at least five endogenous protein markers in said blood sample to obtain a protein profile of said blood sample; and
(c) using said protein profile to determine whether said subject has said disease condition.

39. The method according to claim 38, wherein said quantitating comprises:
(a) contacting said blood sample with an array of a plurality of binding members immobilized on the surface of a solid support under binding conditions, where said plurality includes binding members specific for said at least five endogenous protein markers; and
(b) identifying the amount of each of said at least five endogenous protein markers that binds to its complementary specific binding pair member on said array.

40. The method according to claim 38, wherein determining step (c) comprises comparing said protein profile to a reference.

41. The method according to claim 40, wherein said reference is a reference protein profile.

42. A kit for use in determining whether a subject has a disease condition, said kit comprising:
(a) a means for generating an mRNA expression profile from a disease specific particular blood fraction; and
(b) a means for obtaining a blood sample from a subject.
